(19) United States
(12) Patent Application Publication
(10) Pub. No.: US 2020/0046345 A1
(43) Pub. Date: Feb. 13, 2020

(54) Variable Compression Bone Staple System

(71) Applicants: Thomas Zink, San Antonio, TX (US); Alexandra Zink, San Antonio, TX (US)

(72) Inventors: Thomas Zink, San Antonio, TX (US); Alexandra Zink, San Antonio, TX (US)

(21) Appl. No.: 16/102,035

(22) Filed: Aug. 13, 2018

(51) Int. Cl. A61B 17/064; A61B 17/80; A61B 17/17

(52) U.S. Cl. CPC A61B 17/0642; A61B 2090/0807; A61B 17/17; A61B 17/80

(57) ABSTRACT

A bone staple and integral insertion device that provides biocompatibility, increased strength, and cost effectiveness is disclosed. Once the bone staple is inserted the desired depth into the bone, the arms of the insertion device are spread apart thereby applying the desired amount of compression to the bone staple. The insertion device can then be broken free from the bone staple, and removed from the patient. In this manner, the amount of compression between the broken ends of the bone can be regulated directly by the surgeon during implantation of the bone staple.

### FIELD OF THE INVENTION

The present invention relates to a variable compression bone staple used for the fixation of bone of the musculoskeletal system. More specifically, the invention relates to a bone staple which creates compression in the fractured area of the damaged bone once the bone staple is implanted, and which includes anti-migratory structure for preventing the bone staple from detaching from the bone once implanted. Further, the invention also discloses an insertion device for installing the bone staple in the bone in a precise and timely manner.

### BACKGROUND

Bone staples have been in clinical use for decades and are used in many applications in orthopedics. More specifically, bone staples are generally used to compress broken bone end joints to allow for the fusion of one portion of the bone to another. Early embodiments of bone staples were rather crude and rigid, and comprised of stainless steel or cobalt-chromium U-shaped implants that were commonly hammered into the patient's bone.

Over time, these early bone staples evolved into more modern devices that could be manipulated to compress two adjacent bone segments together. Currently, bone staples are typically implanted by drilling holes into the bone and, upon installation of the bone staple, using heat and/or mechanical means to cause the bone staple to change shape and pull together or compress the damaged bone segments to begin the healing process. Unfortunately, the newer design of bone staple still suffers from a number of limitations. For example, the current bone staple still requires the surgeon to manually manipulate the bone staple while attempting to implant the same into the affected bone, which is both difficult and time consuming. Further, current bone staples, because of their design limitations, require complex and expensive instrumentation, thus impeding their usefulness. For example, if pliers or forceps are used to bend the bone staple to compress the bone segments, the elastic nature of the material that the bone staple is comprised of can cause the bone staple to partially return to its pre-bent shape. Thus, these bone staples may change shape or be manipulated to change shape but do not pull together and compress bone segments with a predictable amount of shape change and compression force.

Additional materials have been utilized to improve upon these deficiencies. For example, most bone staples used today are made of nickel-titanium alloy, or nitinol. Nitinol acquires compression through either the elastic nature of the material or through natural body heat. Nitinol has superior elastic properties, such that if the parallel legs of the bone staple are angled toward the center of the staple and then spread out to be inserted into the bone; the legs of the bone staple will spring back to the center once implanted. However, Nitinol is not an extremely strong or durable material, and it is difficult and, in some cases, impossible to control the amount of compression between the bone segments when using bone staples comprised of Nitinol. Another kind of Nitinol will change shape with ambient temperature. For example, once the Nitinol material reaches body temperature it will compress the legs of the bone staple inward to create compression between the broken ends of the bone. However, these heat sensitive staples are problematic because during implantation the staple can change shape. Furthermore, during shipping, costly strategies must be implemented to keep environmental heating from causing the staple to change shape prior to implantation. For example, strategies such as keeping the staple on dry ice were used to partially overcome this issue but it increased cost and caused the surgeon to have to work quickly in procedures where deliberate, detailed and time-consuming techniques are required to achieve a positive outcome.

Consequently, there is a long felt need in the art for a bone staple that is surgeon controlled and that produces a predictable and desired amount of shape change and compression force. Further, there is a need for a strong, durable bone staple that is also cost effective to manufacture, ship and implant, and that does not change shape prior to implantation causing the surgeon to have to work quickly. The present invention discloses a unique titanium alloy bone staple and integral insertion device that provides biocompatibility, increased strength, and cost effectiveness. The titanium alloy bone staple of the present invention does not materially change shape prior to its implantation, and is also surgeon controlled such that the amount of compression between the broken ends of the bone can be regulated directly by the surgeon.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

In one embodiment, the present invention comprises a unique bone staple that comprises a bridge component having two opposed ends, and a pair of spaced apart legs extending outwardly from either end of the bridge component. Each leg of the bone staple of the present invention further comprises a prong, tab, or protrusion at its proximal end near the bridge component and positioned on the outside or outboard surface of the leg for anti-migratory purposes. Each leg also comprises a tapered angle at a distal end or tip of the leg for compressing the bone segments together upon successful installation. More specifically, the bone staple is inserted into the fractured area of the bone, and secured to the bone segments to create compression forces between the bone segments to bring the segments together to fuse with one another during the healing process. Once implanted, the bridge component of the bone staple is positioned in the cortical bone, and the distal tips of the legs are positioned in the cancellous bone.

In a preferred embodiment of the present invention, an insertion device for installing the bone staple in the patient's bone is integral to the bone staple, and both are manufactured from a titanium alloy, specifically Ti 6 Al 4 V-ELI, though other suitable materials can also be used without affecting the overall concept of the present invention. In one embodiment, the insertion device comprises a pair of arm components with a drill guide component secured to the proximal end of each arm component. Thus, the drill guide components are integral to the inserter and comprise a plurality of serrations on the tip of the drill guides to help grab the bone and prevent slippage during the drilling procedure. Once the drill guides are used, the bone staple is inserted or implanted a desired depth into the bone. A surgeon can then open the insertion device by pulling apart or spreading the arm components to add compression forces to the bone staple. Therefore, the surgeon is able to control the amount of compressive force at the time of implantation of the bone staple. Once the required amount of compression is applied, and the bone staple is inserted in the desired depth into the bone, the insertion device is detached from the bone staple and removed.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

While the specification concludes with claims particularly pointing out and distinctly claiming the present invention, it is believed that the present invention will be better understood from the following description in conjunction with the accompanying FIGS., in which like reference numerals identify like elements, and wherein:

FIG. 1 illustrates a front perspective view of one embodiment of the bone staple of the present invention attached to the insertion device in a closed position and in accordance with the disclosed architecture.

FIG. 2 illustrates a side perspective view of the bone staple and insertion device of the present invention in accordance with the disclosed architecture.

FIG. 3 illustrates a front perspective view of the bone staple and insertion device with arrows showing how the bone staple legs will compress when the arm components of the insertion device are spread apart in accordance with the disclosed architecture.

FIG. 4 illustrates a front perspective view of the bone staple in accordance with the disclosed architecture.

FIG. 5A illustrates a front perspective view of the bone staple and insertion device in a closed position in accordance with the disclosed architecture.

FIG. 5B illustrates a front perspective view of the bone staple and insertion device in an open position in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

Generally stated, the present invention relates to a titanium alloy bone staple and integral insertion device that provides biocompatibility, increased strength, and cost effectiveness. The titanium alloy bone staple does not change shape prior to implantation, and does not require the elaborate or expensive shipping or storage environments associated with prior art bone staples. Once the bone staple is inserted to the desired depth into the bone, the arms of the insertion device are spread apart to apply the desired amount of compression to the bone staple and, ultimately, to the bone segments. Then, the insertion device is detached from the bone staple and removed from the patient. In this manner, the amount of compression between the broken segments of the bone can be regulated directly by the surgeon. Further, anti-migration features on the bone staple prevent the bone staple from detaching from the bone.

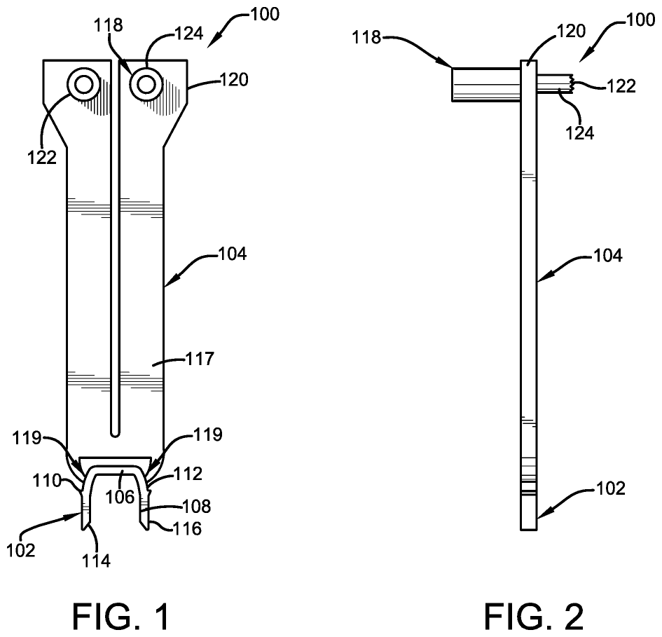

Referring initially to the drawings, FIG. 1 illustrates a perspective view of one embodiment of the bone staple system 100 of the present invention, and FIG. 2 illustrates a side perspective view of the bone staple system of FIG. 1 in accordance with the disclosed architecture. The bone staple system 100 is preferably comprised of a bone staple 102 and an insertion device 104. As explained more fully below, the bone staple 102 may be integrally formed with, but eventually detachable from, the insertion device 104, and both are preferably comprised of a titanium alloy, specifically Ti 6 Al 4 V-ELI, though other suitable medical grade materials can also be used without affecting the overall concept of the present invention.

The bone staple 102 is preferably comprised of a bridge component 106 and a pair of spaced apart legs 108 extending outwardly from the bridge component 106 and the bone staple system 100 overall. More specifically, a leg 108 is positioned at each end of bridge component 106, and extends outwardly therefrom for insertion into a patient's bone (not shown). As best shown in FIGS. 1-4, one or both of the legs 108 preferably comprise a prong, tab, or protrusion 110 at its proximal end 112 and near the bridge component 106. Protrusions 110 permit the bone staple 102 to better grip the bone segments once implanted into a patient (not shown). More specifically, the prong, tab, or protrusion 110 on the legs 108 provide anti-migration features for the bone staple 102 once it is implanted into the fractured area of the bone. Each leg 108 of bone staple 102 also comprises a tapered angle 114 at a distal end or tip 116 of the leg 108, thereby allowing for easier insertion into the patient's bone and for compressing the bone segments together to fuse during the healing process. More specifically, the tapered angle 114 acts to better engage the bone during insertion of the bone staple 102, pushing the legs 108 together.

Notwithstanding, one of ordinary skill in the art will appreciate that the shape and size of the bone staple 102, as shown in FIGS. 1-4, are for illustrative purposes only, and that many other shapes and sizes of bone staple 102 are well within the scope of the present disclosure. Although the particular dimensions of the bone staple 102 (i.e., length, width, and height) are important design parameters for good performance, the bone staple 102 may be any shape or size that ensures optimal performance during use and is within the overall objective of the present invention.

As previously discussed, the bone staple 102 is preferably comprised of a titanium alloy, and most preferably of Ti 6 Al 4 V-ELI, though it is contemplated that other medical grade materials can also be used without affecting the overall concept of the present invention. More specifically, bone staple 102 is preferably laser cut from a single piece of titanium alloy, specifically Ti 6 Al 4 V-ELI, thereby lowering the cost of manufacturing bone staple 102. While laser cutting is the preferred method of manufacturing bone staple 102, other manufacturing techniques are also contemplated such as casting, molding, forming, additive manufacturing, etc. Similarly, it is also contemplated that the various components of the bone staple 102 could be manufactured separately and then joined together prior to use.

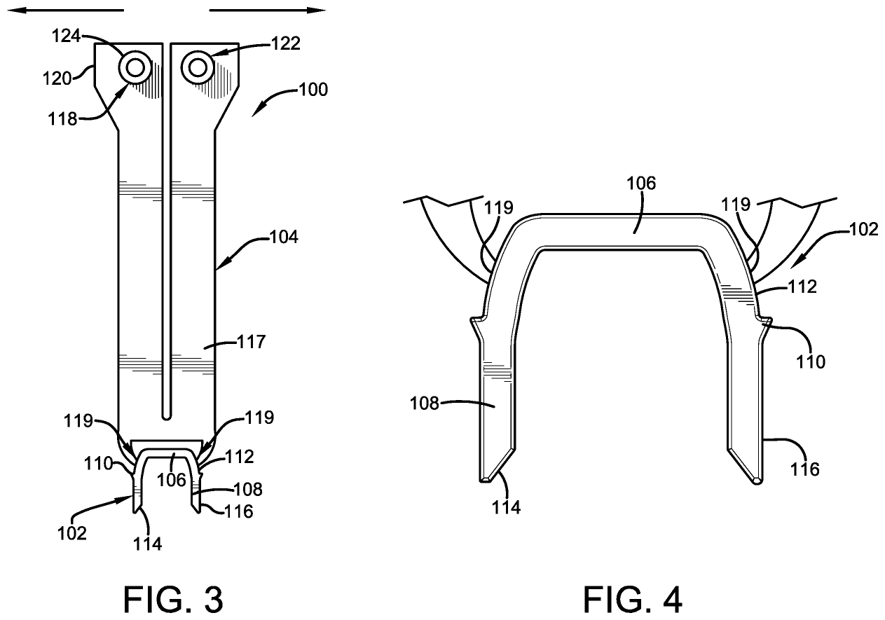

As best shown in FIGS. 1-3, 5A and 5B, insertion device 104 is preferably integral to, and eventually detachable from, the bone staple 102. Insertion device 104 is preferably comprised of a pair of spaced apart arm components 117 that pivot about a pivot point on insertion device 104 nearest bone staple 102. Accordingly, a surgeon can open the insertion device 104 by pulling apart (spreading) the arm components 117 to add compression to the bone staple 102. In this manner, the surgeon is able to control the amount of compressive force at the time of implantation of the bone staple 102 into the patient's bone (not shown) with the bone staple system 100. FIG. 3 illustrates a front perspective view of the bone staple 102 and insertion device 104 with arrows showing how the bone staple legs 108 will compress when the arm components 117 of the insertion device 104 are spread apart (i.e., pulled in opposite directions).

Each of the pair of arm components 117 are secured to a respective leg 108 of the bone staple 102 at a break-off point 119. Thus, once the bone staple 102 is implanted to the desired depth into the patient's bone, the insertion device 104 is moved up and down and/or rotated until the break-off points 119 weaken and break. In this manner, the insertion device 104 is separated from the bone staple 102 and removed from the patient.

Further, the arm components 117 also comprise a drill guide component 118 secured to the proximal end 120 of each arm component 117. Thus, the drill guide components 118 are integral to the insertion device 104. As best shown in FIGS. 1-4, the drill guide components 118 preferably comprise a plurality of serrations 122 on the tip 124 of the drill guide components 118 so as to better grip the bone segments and prevent slipping during the drilling procedure. More specifically, the plurality of serrations 122 on the drill guide components 118 provide anti-migration features for the drill guide components 118 relative to the patient's bone during the drilling procedure.

Notwithstanding, one of ordinary skill in the art will appreciate that the shape, size, and configuration of the insertion device 104, as shown in FIGS. 1-4, is for illustrative purposes only and that many other shapes, sizes and configurations of the insertion device 104 are well within the scope of the present disclosure. Although dimensions of the insertion device 104 (i.e., length, width, and height) are important design parameters for good performance, the insertion device 104 may be any shape or size that ensures optimal performance during use and is within the overall objective of the present invention.

The insertion device 104 is preferably laser cut from a single piece of titanium alloy, specifically Ti 6 Al 4 V-ELI, though other suitable materials can also be used without affecting the overall concept of the present invention. In a preferred embodiment, the insertion device 104 is integral to the bone staple 102, and both are manufactured from and laser cut from a single piece of titanium alloy, specifically Ti 6 Al 4 V-ELI. In another embodiment, the bone staple 102 and insertion device 104 of the present invention may be integrally manufactured using additive manufacturing techniques, or by using a combination of other molding or machining techniques (e.g., injection molding, machining, etc.) to produce the subject bone staple and insertion device. These additional techniques include, without limitation, material extrusion, vat photo polymerization, powder bed fusion, material jetting, binder jetting, sheet lamination and directed energy deposition, or any other suitable technique as is known in the art.

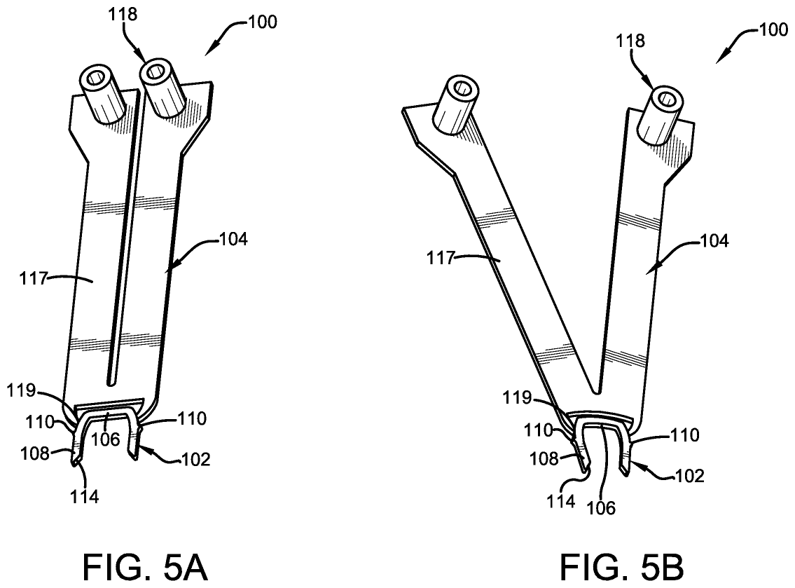

Having described a preferred embodiment of the bone staple system 100, its use will now be described in general terms. During surgical insertion of the bone staple system 100 into a patient (not shown), the bone staple 102 is inserted into the fractured area (osteotomy) of the bone and secured to the bone segments to create compression between the bone segments. Once implanted, the bridge component 106 of the bone staple 102 is positioned in the cortical bone and the distal tips 116 of the legs 108 are positioned in the cancellous bone. More specifically, and with the insertion device in a closed position as best illustrated in FIG. 5A, a surgeon utilizes the drill guide components 118 to drill holes into the fractured area of the bone to guide insertion of the bone staple 102. One of ordinary skill in the art will appreciate that the spacing between drill guide components 118 matches the spacing between legs 108 of bone staple 102 when the insertion device is in the closed position.

Once the drill guide components 118 are used and the guide holes are created, the bone staple 102 is inserted (implanted) the desired depth into the bone either by surgeon force or by mallet or other instrument persuasion. As best shown in FIG. 5B, once the bone staple 102 is inserted to the desired depth, a surgeon can then open the inserter 104 by pulling apart (spreading) the arm components 117 to add compression to the bone staple 102. In this manner, the surgeon is able to control the amount of compressive force at the time of implantation with the bone staple system 100. Once the required amount of compression is applied, and the bone staple 102 is inserted in the desired depth into the bone, the insertion device 104 is broken free from the bone staple 102 and removed. More specifically, the insertion device 104 is broken free from the bone staple 102 by moving the insertion device 104 up and down or rotating the same until the break-off points 119 weaken and break. Once the break-off points 119 are weakened, the inserter 104 is then broken free from the bone staple 102 and removed.

Accordingly, the bone staple system 100 of the present invention delivers a bone staple 102 that is surgeon controlled and that produces a predictable and desired amount of shape change and compression force, along with anti-migratory features that prohibit the bone staple 102 from being inadvertently removed from the bone once implanted. Further, the durable bone staple 102 of the present invention is cost effective to manufacture, ship and implant, and does not change shape prior to implantation like other prior art bone staples. Additionally, the bone staple system 100 of the present invention further comprises an integrally formed insertion device 104 that permits the surgeon to quickly and precisely implant the bone staple 102, and then separate the insertion device 104 from the bone staple 102, and remove the same from the patient.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A bone staple system comprising:
a bone staple; and
an insertion device integral to the bone staple and comprising a pair of arm components, wherein the pair of arm components are repositionable to apply compression to the bone staple during implantation.

2. The system of claim 1, wherein the bone staple comprises a bridge component and a pair of legs, wherein said pair of legs further comprise an anti-migratory protrusion at a proximal end of said pair of legs.

3. The system of claim 2, wherein the pair of legs of the bone staple further comprises a tapered angle at a distal tip of the pair of legs.

4. The system of claim 1, wherein both the bone staple and the insertion device are laser cut from a single piece of titanium alloy.

5. The system of claim 4, wherein the titanium alloy is Ti 6 Al 4 V-ELI.

6. The system of claim 1, wherein the pair of arm components comprise a break-off point at their proximal end.

7. The system of claim 6, wherein the bone staple is detachable from the insertion device at the break-off point.

8. The system of claim 1, wherein the pair of arm components further comprises a drill guide component secured to a proximal end of each of said pair of arm components.

9. The system of claim 8, wherein the drill guide component comprises a plurality of serrations on a tip of the drill guide component.

10. The system of claim 1, wherein the bone staple and the insertion device are integrally manufactured using additive manufacturing techniques.

11. The system of claim 2, wherein once the bone staple is implanted, the bridge component is positioned in cortical bone.

12. The system of claim 2, wherein once the bone staple is implanted, the pair of legs are positioned in cancellous bone.

13. A bone staple comprising:
a bridge component;
a pair of legs, wherein a single leg of the pair of legs is secured to either end of the bridge component; and
an integrally formed insertion device; and
wherein once the bone staple is implanted into a bone, the bridge component is positioned in a cortical portion of the bone and the pair of legs is positioned in a cancellous portion of the bone; and
wherein the integrally formed insertion device applies compression to the bone staple during implantation.

14. The bone staple of claim 13, wherein the integrally formed insertion device comprises a pair of repositionable arms which apply a compressive force to the bone staple when the pair of arms are spread apart.

15. The bone staple of claim 13, wherein the pair of legs comprise a protrusion at a proximal end of said pair of legs.

16. The bone staple of claim 13, wherein the pair of legs further comprises a tapered angle at a distal tip of said pair of legs.

17. The bone staple of claim 13, wherein the bone staple is laser cut from a single piece of titanium alloy.

18. The bone staple of claim 17, wherein the titanium alloy is Ti 6 Al 4 V-ELI.

19. The method of implanting a bone staple using a bone staple system comprising:
utilizing drill guide components on an insertion device to drill a pair of guide holes into a fractured bone area;
inserting a bone staple into the pair of guide holes via a pair of legs on the bone staple;
pulling apart a pair of arm components on the insertion device to add a compressive force to the bone staple; and
separating the bone staple from the insertion device at a break-off point.

20. The method of claim 19 wherein the insertion device is integral to the bone staple and both are manufactured of a titanium alloy.
